(19) United States
(12) Patent Application Publication
(10) Pub. No.: US 2015/0017227 A1
(43) Pub. Date: Jan. 15, 2015

(54) Methods and Compositions for Treating Skin Diseases and Conditions

(71) Applicants: Epitop, Inc., Poway, CA (US); National Jewish Health, Denver, CO (US)

(72) Inventors: Byung Eui Kim, Englewood, CO (US); Donald Y.M. Leung, Englewood, CO (US)

(21) Appl. No.: 14/329,321

(22) Filed: Jul. 11, 2014

Related U.S. Application Data
(60) Provisional application No. 61/844,972, filed on Jul. 11, 2013.

(51) Int. Cl. A61K 31/222; A61K 31/045; A61K 31/16; A61K 31/015; A61K 31/20; A61K 31/05

(52) U.S. Cl. CPC A61K 31/222; A61K 31/20; A61K 31/05; A61K 31/16; A61K 31/015; A61K 31/045
USPC 424/450; 514/546; 514/558; 514/729; 514/627; 514/661; 560/231; 554/1; 568/819; 568/733; 554/35; 585/24; 585/27; 585/23; 585/20; 585/22

(57) ABSTRACT

Disclosed are methods and compositions for treating skin diseases or conditions with a composition containing a lipid component as an active ingredient.

### CROSS-REFERENCE TO RELATED APPLICATIONS

This application claims the benefit of priority under 35 U.S.C. §119(e) from U.S. Provisional Application No. 61/844,972, filed Jul. 11, 2013. The entire disclosure of U.S. Provisional Application No. 61/844,972 is incorporated herein by reference.

### FIELD OF THE INVENTION

The present invention generally relates methods and compositions for treating skin diseases or conditions with a composition containing a lipid component as an active ingredient.

### BACKGROUND OF THE INVENTION

The skin plays a key role in protecting animals including humans against environmental factors. The most important barrier function exists in the epidermis which is the outermost layer of the skin and protects the skin from various external stimuli (physical and chemical stimuli such as chemicals, pollutants, dry environment, microbes, allergens and UV radiation) and prevents excessive loss of water through the skin. This protective function can be maintained only when the keratinocytes undergo normal function. The horny layer (stratum corneum), the outermost layer of the epidermis, is formed from keratinocytes and consists of terminally differentiated keratinocytes surrounded by lipid layers. Keratinocytes are the cells generated as a result of the process in which basal cells that continuously proliferate in the lowest layer of the epidermis move up toward the skids surface while they undergo a series of structural and functional changes. After a given period, old keratinocytes are shed from the skin and replaced by new keratinocytes. This repeated process is called “differentiation of epidermal cells” or “keratinization”. During the keratinization process, keratinocytes form the horny layer, while they produce natural moisturizing factors (NMFs) intercellular lipids (ceramides, cholesterols and fatty acids), such that the horny layer has firmness and softness to function as a skin barrier. Keratinocytes also differentiate to produce skin barrier or structural proteins such as filaggrin, involucrin and loricrin as well as antimicrobial peptides (e.g. human beta defensins or HBD) needed to kill invading microbes and attract inflammatory cells involved in host defense.

However, this horny layer can easily lose its functions due to lifestyle factors such as excessive face washing or bathing, environmental factors such as dry atmosphere or pollutants, and immunologic responses found in allergic skin disease or aging. In fact, due to various factors which have increased recently, more and more people are suffering from dry skin symptoms and various skin barrier disorders.

### SUMMARY OF THE INVENTION

One embodiment of the invention relates to a method for treating a skin disease or condition in an animal, the method comprising administering to the animal a composition comprising a lipid component selected from the group consisting of alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal and combinations thereof, as an active ingredient.

In one aspect the lipid component comprises terpinyl acetate, guaiol, elemol, sabinene, palmitic acid, thujopsene, totarol, 9-octadecenamide, β-pinene and cembrene. In still another aspect, the lipid component consists essentially of terpinyl acetate, guaiol, elemol, sabinene, palmitic acid, thujopsene, totarol, 9-octadecenamide, β-pinene and cembrene. In still another aspect, the lipid component consists of terpinyl acetate, guaiol, elemol, sabinene, palmitic acid, thujopsene, totarol, 9-octadecenamide, β-pinene and cembrene.

The skin disease or condition can be an inflammatory skin disease or condition. In one aspect, the inflammatory skin disease can be atopic dermatitis, contact dermatitis, seborrheic dermatitis, and acne. In still another aspect, the skin disease or condition can be selected from xeroderma, atopic dermatitis, contact dermatitis, psoriasis, and ichthyosis. In yet another aspect, the skin disease or condition is a skin wound.

In another aspect, the lipid component induces expression of filaggrin. In still another aspect, the lipid component induces expression of beta-defensin-3 (HBD-3). In yet another aspect, the lipid component attenuates the inhibitory effect of proinflammatory cytokines on filaggrin expression or on beta-defensin-3 expression. In one aspect, the proinflammatory cytokine is a Th2 cytokine. In still another aspect, the Th2 cytokines can be IL-4, IL-13, IL-25, IL-31 or IL-33. In addition, the lipid component reduces LDH release, is non-toxic and protects keratinocyte.

Another embodiment of the invention relates to a method of inducing expression of filaggrin and/or beta-defensin in keratinocytes of an animal in need thereof comprising administering to the animal a composition comprising a lipid component selected from the group consisting of alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal and combinations thereof as an active ingredient, wherein the lipid component induces expression of filaggrin, as an active ingredient, wherein the lipid component induces expression of filaggrin and/or beta-defensin.

Another embodiment of the invention relates to a method for preventing a skin disease or condition in an animal, the method comprising administering to the animal a composition comprising a lipid component selected from the group consisting of alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal and combinations thereof as an active ingredient and wherein the skin disease or condition is skin cancer or skin burn from UV exposure.

In the methods of present invention, the composition comprising the lipid component can be administered topically or transdermally. In still other aspects, the composition is in a form selected from a solution, a gel, a solid, a dough anhydride, an emulsion, a suspension, a microemulsion, microcapsules, microgranules, ionic (liposome) and non-ionic vesicles, cream, skin lotion, an ointment, powder, a spray, a conceal stick, foam and aerosol. In still other aspects, the composition is administered to the animal in an amount effective to treat the skin disease or condition.

Further, in the methods of the present the beta-defensin can be human beta-defensin3 (HBD-3) and still further, the animal can be human.

Another embodiment of the invention is a pharmaceutical composition comprising a pharmaceutically-acceptable carrier, and a Chamaecyparis obtusa lipid component. In one aspect the Chamaecyparis obtusa lipid component cane be selected from alpha-thujene, alpha-pinene, camphene, sabinene, beta-pinene, alpha-terpinene, benzene, limonene, peltay3-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal and combinations thereof.

Another embodiment of the invention is a lipid fraction from Chamaecyparis obtusa. In one aspect, the fraction comprises one or more of the following compounds alpha-thujene, alpha-pinene, camphene, sabinene, beta-pinene, alpha-terpinene, benzene, limonene, peltay3-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal and combinations thereof. In a preferred aspect, the lipid fraction comprises one or more of the following lipids selected from the group consisting of terpinyl acetate, guaiol, elemol, sabinene, palmitic acid, thujopsene, totarol, 9-octadecenamide, β-pinene and cembrene. In aspects of the invention, the lipids are synthetically produced. In other aspects, the lipids are extracted from the Chamaecyparis obtusa plant.

### BRIEF DESCRIPTION OF THE DRAWINGS

FIG. 1 shows filaggrin gene expression in undifferentiated human keratinocytes treated with a lipid component comprising the following lipids alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal.

FIG. 2 shows filaggrin gene expression in differentiated human keratinocytes treated with a lipid component comprising the following lipids alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal.

FIG. 3 shows human 3-defensin-3 (HBD-3) gene expression in undifferentiated human keratinocytes treated with a lipid component comprising the following lipids alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal.

FIG. 4 shows human β-defensin-3 gene expression in differentiated human keratinocytes treated with a lipid component comprising the following lipids alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal.

FIG. 5 shows 0.001% of a lipid component comprising the following lipids alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal, overcomes inhibitory effects of Th2 cytokines on filaggrin expression in undifferentiated keratinocytes.

FIG. 6 shows 0.001% of a lipid component comprising the following lipids alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal overcomes inhibitory effects of Th2 cytokines on filaggrin expression in differentiated keratinocytes.

FIG. 7 shows 0.001% of a lipid component comprising the following lipids alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal overcomes inhibitory effects of Th2 cytokines on β-defensin-3 expression in differentiated keratinocytes.

FIG. 8 shows that a lipid component comprising the following lipids alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal does not have keratinocyte toxicity.

FIGS. 9A and 9B show lipid component comprising the following lipids: terpinyl acetate, guaiol, elemol, sabinene, palmitic acid (also referred to as hexadecanoic acid), thujopsene (also referred to as widdrene), totarol, 9-octadecenamide, β-pinene and cembrene, induces filaggrin (FIG. 9A) and HBD-3 (FIG. 9B) gene expression. Human primary keratinocytes were stimulated with various concentrations of 10 purified lipid mixture for 2 days. The gene expression of filaggrin and HBD-3 was examined using real-time RT-PCR. As shown in FIG. 9A filaggrin gene expression was significantly (P<0.001) induced by the purified lipid mixture with concentration as low as 0.01% compared with media alone. As shown in FIG. 9B HBD-3 gene expression was significantly (P<0.05) induced by the lipid mixture with a concentration as low as 0.01% compared with media alone.

FIGS. 10A and 10B show that a lipid component comprising the following lipids: terpinyl acetate, guaiol, elemol, sabinene, palmitic acid (also referred to as hexadecanoic acid), thujopsene (also referred to as widdrene), totarol, 9-octadecenamide, β-pinene and cembrene, overcomes inhibitory effects of Th2 cytokines on filaggrin (FIG. 10A) and HBD-3 (FIG. 10B) gene expression. Human primary keratinocytes were incubated with 50 ng/mL of IL-4 and 50 ng/mL of IL-13 for a day, and then the cells were continuously stimulated with IL-4 and IL-13 in the presence or absence of 0.02% of the 10 purified lipid mixture for an additional 2 days. The gene expression of filaggrin and HBD-3 was examined using real-time RT-PCR. FIG. 10A shows that filaggrin gene expression was significantly (P<0.05) increased in keratinocytes treated with a combination of Th2 cytokines and 0.02% of the lipid component compared with keratinocytes treated Th2 cytokines alone. FIG. 10B shows HBD-3 gene expression was significantly (P<0.01) increased in keratinocytes treated with a combination of Th2 cytokines and 0.02% of the lipid component compared with keratinocytes treated Th2 cytokines alone.

FIGS. 11A and 11B show that a lipid component comprising the following lipids: terpinyl acetate, guaiol, elemol, sabinene, palmitic acid (also referred to as hexadecanoic acid), thujopsene (also referred to as widdrene), totarol, 9-octadecenamide, β-pinene and cembrene, inhibits Staphylococcus aureus. The inhibitory effects of the purified lipid mixture on bacteria were analyzed using bactericidal assay. As shown in FIG. 11A methicillin sensitive S aureus (MSSA) was significantly (P<0.001) inhibited by the lipid component with a concentration as low as 0.01%. As shown in FIG. 11B methicillin resistant S aureus (MRSA) was significantly (P<0.001) inhibited by the lipid component with a concentration as low as 0.01%.

FIG. 12 shows that a lipid component comprising the following lipids: terpinyl acetate, guaiol, elemol, sabinene, palmitic acid (also referred to as hexadecanoic acid), thujopsene (also referred to as widdrene), totarol, 9-octadecenamide, β-pinene and cembrene, is not toxic to human primary keratinocytes. The toxicity of the lipid component on keratinocytes was evaluated with lactate dehydrogenase (LDH) assay. LDH release was not increased by the purified lipid mixture as high as 0.04%, but reduced by various concentrations of the lipid component, which means the lipid mixture has a protective effect for keratinocyte.

### DETAILED DESCRIPTION OF THE INVENTION

This invention generally relates to methods and compositions for treating skin diseases or conditions in an animal with a lipid component. The lipid component can be selected from one or more of the following lipids alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal and combinations thereof.

As referred to herein a lipid component refers to one or more lipids, up to and including all of the following lipids alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal as well as combinations of the lipids. The lipid component can comprise nineteen, eighteen, seventeen, sixteen, fifteen, fourteen, thirteen, twelve, eleven, ten, nine, eight, seven, six, five, four, three, two, or one of the lipids. For example, the lipid component can comprise terpinyl acetate, guaiol, elemol, sabinene, palmitic acid (also referred to as hexadecanoic acid), thujopsene (also referred to as widdrene), totarol, 9-octadecenamide, β-pinene and cembrene.

The lipid component can be extracted from Chamaecyparis obtusa, wherein the lipid component can be a single lipid or a combination of two or more lipids that are present in a Chamaecyparis obtusa lipid extract. A Chamaecyparis obtusa lipid component can be prepared by making a whole Chamaecyparis obtusa lipid extract. The lipid component of the present invention can be prepared from a Chamaecyparis obtusa lipid extract from any part of a Chamaecyparis obtuse tree (including without limitation, whole tree, leaves, bark, trunk, branches and root) that includes lipid components. Chamaecyparis obtusa lipid extracts can be prepared by methods known in the art for separating, purifying or recovering lipids from more complex mixtures. For example, Example 1 presented herein, describes one such method for preparing a Chamaecyparis obtusa lipid extract by supercritical fluid extraction. Alternatively, a lipid component can be a portion or a fraction of a whole Chamaecyparis obtusa lipid extract.

Preferably, the lipid component can be prepared by combining one or more lipids that are found in a Chamaecyparis obtusa lipid extract, where the lipids are purchased or synthesized and combined synthetically. Additionally, one or more of the lipids of the lipid component are chemically and/or synthetically synthesized by methods known to those of skill in the art.

Chamaecyparis obtusa that is used in the present invention is an evergreen tall tree belonging to the family Cupressaceae and grows to a height of 50 m and a diameter of 2 m. Its branches spread horizontally to form a conical crown, and its bark is red brown in color and splits vertically and exfoliates. It grows straight so that it has a very wide range of applications. It is mainly used in construction materials, civil engineering materials, ships, or chip materials.

Without being bound by theory, the present inventors have found the surprising result that a lipid component comprising alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal, induces the skin barrier protein, filaggrin, enhances the expression of the antimicrobial peptide, HBD-3 and attenuates the inhibitory effect of proinflammatory cytokines on filaggrin and/or beta-defensin 3 expression. The proinflammatory cytokines include but are not limited to Th2 cytokines such as IL-4, IL-13, IL-25, IL-31 and IL-33 as well as, IL-1, TNF-alpha, IL-6 and IL-22.

In addition, without being bound by theory, the present inventors have found the surprising result that a lipid component comprising terpinyl acetate, guaiol, elemol, sabinene, palmitic acid (also referred to as hexadecanoic acid), thujopsene (also referred to as widdrene), totarol, 9-octadecenamide, β-pinene and cembrene, induces the skin barrier protein, filaggrin, enhances the expression of the antimicrobial peptide, HBD-3 and attenuates the inhibitory effect of proinflammatory cytokines on filaggrin and/or beta-defensin 3 expression. The proinflammatory cytokines include but are not limited to Th2 cytokines such as IL-4, IL-13 and IL-25, as well as, IL-1, TNF-alpha, and IL-6. Furthermore, the lipid mixture reduces LDH release from keratinocyte, suggesting the lipid mixture is not toxic to keratinocyte and has a protective effect for keratinocyte.

This invention includes methods comprising administering a composition containing a lipid component disclosed herein which induces expression of filament aggregating protein (herein referred to as filaggrin) and/or beta-defensin, including beta-defensin-3. The lipid component is an active ingredient which induces the expression of filaggrin and/or beta-defensin.

The epidermis provides a physical and permeability barrier, which protects the skin from invasion of microbes and allergens. Filaggrin is a key epidermal barrier protein, and is downregulated by Th2 cytokines such as interleukin (IL)-4 and IL-13 in AD skin. Deficiency of filaggrin allows enhanced penetration of microbes and allergens through the epidermal barrier of AD, psoriasis and contact dermatitis as compared to normal skin. The epidermis also produces several antimicrobial peptides (AMPs), which prevent microbial skin infections by bacteria and viruses. Human beta defensin (HBD)-3 is the most important AMP in epidermis to prevent skin infection. However, HBD-3 is decreased in AD skin by Th2 cytokines. Recurrent skin infections by microbes such as staphylococcus aureus and herpes simplex virus are common complications in AD. Therefore, filaggrin and HBD-3 play an important role in the epidermis to prevent skin infection and to maintain a healthy skin condition.

Filaggrin induction has also recently been shown to be beneficial in protecting against sunburns because it is known to absorb the harmful effects of ultraviolet irradiation. As such it may reduce the risk of developing skin cancer from UV exposure (Uddin, A., et al. Toxicology and Applied Pharmacology 265 (2012) 335-341). Subjects with reduced filaggrin in their skin may therefore be more susceptible to skin cancer and increased filaggrin expression may prevent skin cancer.

Various skin diseases or skin conditions can be treated by the methods and compositions of the present invention, including but not limited to xeroderma, atopy, psoriasis, and ichthyosis vulgaris, as well as inflammatory skin diseases and skin conditions such as atopic dermatitis, contact dermatitis, seborrhoic dermatitis, and acne as well as other filaggrin deficient conditions including but not limited to asthma and allergic rhinitis associated with atopic dermatitis. Additionally, the methods and compositions of the present invention can be effective for use in skin anti-aging, reducing skin wrinkles, improving skin elasticity, skin whitening, skin moisturizing, preventing and amelioration of dry skin diseases, anti-inflammation, skin regeneration, UV skin protection, skin sunburn protection, and skin cancer prevention.

Still further, the methods and compositions of the present invention can treat a skin wound. The skin also functions as a barrier that protects the body from the external environment. When the skin is wounded, the site of the wound site is filled with blood and neighboring skin resident cells by natural healing action that results in a wound healing process taking place.

Skin disease refers to all disorders occurring on the skin of animals including humans. Inflammatory skin disease refers to a disease that involves a series of clinical signs and symptoms, such as itching, edema, erythema and stripping, due to various stimulating factors which cause a series of inflammatory reactions in the skin epidermis. Known inflammatory skin diseases include atopic dermatitis, contact dermatitis, seborrhoic dermatitis, acne, psoriasis, aging skin, etc.

For the treatment of the inflammatory skin diseases, antihistamine agents, vitamin ointments and corticosteroids, as well as anti-inflammatory calcineurin inhibitors have been used to date. However, such drugs mostly have temporary effects and can have severe side effects in some cases.

Atopic dermatitis (AD) is a chronic inflammatory skin disorder that affects up to 20% of children and places a heavy economic burden on patients and their families Current treatment of AD includes antibiotics, corticosteroids and calcineurin inhibitors such as pimecrolimus and tacrolimus. However, frequent use of antibiotics can produce multi-drug resistant organisms such as methicillin-resistant staphylococcus aureus (MRSA) and vancomycin-resistant enterococci (VRE), and prolonged use of topical steroids and calcineurin inhibitors can cause adverse side effects including skin atrophy, tachyphylaxis, steroid rosacea, skin burning, pruritus, skin erythema, immunosuppression, lymphoma and skin cancer. In addition, calcineurin inhibitors is not FDA approved to be prescribed under age of 2 due to its potential side effects although AD is very common in young infants. Therefore, safe and effective new drugs are required for patients with AD particularly young children.

Subjects with inflammatory skin diseases such as atopic dermatitis, can suffer from recurrent skin infections and inflammation due to epidermal barrier defects, such as those caused by a lack of filaggrin, and antimicrobial peptide deficiency, such as by a reduction in human beta defensin (HBD)-3. In addition, Th2 cytokines, which are known to be overexpressed in the skin of those with inflammatory skin diseases such as atopic dermatitis, inhibit production of the epidermal barrier protein and antimicrobial peptides.

In addition to treating skin diseases, the methods and compositions of the present invention can be used for treating allergic rhinitis, food allergies, intestinal allergies, as well as allergies associated with abnormal epithelial cell barrier and/or microbiome abnormalities, including inflammatory bowel disease.

Mutations in the filaggrin gene (FLG) are among the most common and profound single-gene defects identified to date in the causation and modification of disease. FLG encodes an important epidermal protein abundantly expressed in the outer layer of the epidermis. The critical role of filaggrin in epidermal function underlies the pathogenic importance of this gene in common dermatologic and allergic diseases. FLG mutation carriers have a greatly increased risk of common complex traits, including atopic dermatitis, contact allergy, asthma, hay fever and peanut allergy. (Irvine, A. D., et al. Filaggrin Mutations Associated with Skin and Allergic Diseases; The New England Journal of Medicine 365; 14, 1315-1327; 2011)

Mammalian and/or human beta-defensins are antimicrobial peptides implicated in the resistance of epithelial surfaces to microbial colonization and infection. They are produced by keratinocytes and neutrophils. They can be downregulated immune cytokines (e.g. IL-4, IL-13, IL-25) found in allergic diseases such as atopic dermatitis and they are increased in keratinocytes when they undergo differentiation.

The composition comprising the lipid component of the present invention can be administered by an administration route including but not limited to topical, transdermal, oral and nasal administration routes. Dosage forms for topical administration or for transdermal administration of the lipid component of the invention include powders, sprays, ointments, pastes, creams, lotions, gels, solutions, patches, drops and inhalants. The active ingredient may be mixed under sterile conditions with a pharmaceutically-acceptable carrier, and with any buffers, or propellants which may be required. The carriers, buffers and propellants may be non-naturally occurring. The ointments, pastes, creams and gels may contain, in addition to the active ingredient, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to the active ingredient, excipients such as lactose, talc, silicic acid, aluminum hydroxide, calcium silicates and polyamide powder or mixtures of these substances. The excipients can be non-naturally occurring excipients. Sprays can additionally contain customary propellants such as chlorofluorohydrocarbons and volatile unsubstituted hydrocarbons, such as butane and propane. Transdermal patches have the added advantage of providing controlled delivery of compounds of the invention to the body. Such dosage forms can be made by dissolving, dispersing or otherwise incorporating one or more compounds of the invention in a proper medium, such as an elastomeric matrix material. Absorption enhancers can also be used to increase the flux of the compound across the skin and can be non-naturally occurring. The rate of such flux can be controlled by either providing a rate-controlling membrane or dispersing the compound in a polymer matrix or gel. A drug-impregnated solid carrier (e.g., a dressing) can also be used for topical administration and can be non-naturally occurring.

The administration dose of the lipid component which is the active ingredient of the pharmaceutical composition may vary depending on the age, sex and bodyweight of the subject in need of treatment, the particular disease to be treated or pathological conditions thereof, severity of the disease or pathological conditions, administration route, and discretion of a physician or pharmacist. The administration dose may be determined by those skilled in the art in consideration of those factors and is an amount effective to treat the skin disease or condition.

The composition can be for external skin application and can contain cosmetically and skin-scientifically acceptable medium or base. The composition may be formulated as a preparation for local application. Examples of formulations for local application include a solution, a gel, a solid, a dough anhydride, an emulsion prepared by dispersing an oil phase in a water phase, a suspension, a microemulsion, microcapsules, microgranules, ionic (liposome) and non-ionic vesicles, cream, skin lotion, an ointment, powder, a spray, and a conceal stick. In addition, the composition of the present invention can be formulated according to a conventional method known in the art. Also, the composition for external skin application according to the present invention can be formulated as a foam composition or an aerosol composition further containing a compressed propellant.

The composition for skin external application according to the present invention may contain additives which are conventionally field in the cosmetic field or the skin science field, for example, a fatty substance, an organic solvent, a solubilizing agent, a thickener, a gelling agent, a softener, an antioxidant, a suspending agent, a stabilizer, a foaming agent, an aromatic, a surfactant, water, an ionic or non-ionic emulsifying agent, a filler, a sequestering agent, a chelating agent, a preservative, vitamins, a blocker, a moisturizing agent, essential oil, a dye, a pigment, a hydrophilic or hydrophobic activator, a lipid vesicle, or other components which are generally used in cosmetics. These can be non-naturally occurring additives. The pharmaceutical composition may contain pharmaceutical additives such as antiseptics, stabilizing agents, hydrating agents, emulsification promoters or salts and/or buffers for osmotic control and may further contain other therapeutically useful substances. The pharmaceutical composition may be formulated into lotion, cream, ointment, gel, or the like.

The topical formulations can also include absorption enhancers, permeation enhancers, thickening agents, viscosity enhancers, agents for adjusting and/or maintaining the pH, agents to adjust the osmotic pressure, preservatives, surfactants, buffers, salts (preferably sodium chloride), suspending agents, dispersing agents, solubilizing agents, stabilizers and/or tonicity agents. These additives are contained in amounts which are generally used in the cosmetic field or the skin science field.

There is no particular limitation on the formulation of the inventive composition for skin external application containing the lipid component. It may be formulated into cosmetic products, for example, skin lotion, astringent lotion, milk lotion, nourishing cream, massage cream, essence, eye cream, eye essence, cleansing cream, cleansing foam, cleansing water, pack, powder, body lotion, body cream, body oil and body essence.

While it is possible for the lipid component of the present invention to be administered alone, it is preferable to administer the compound as a pharmaceutical formulation (composition). The pharmaceutical compositions of the invention comprise a lipid compound or compounds of the invention as an active ingredient in admixture with one or more pharmaceutically-acceptable carriers and, optionally, with one or more other compounds, drugs or other materials. Each carrier must be “acceptable” in the sense of being compatible with the other ingredients of the formulation and not injurious to the animal. Pharmaceutically-acceptable carriers are well known in the art. Regardless of the route of administration selected, the compounds of the present invention are formulated into pharmaceutically-acceptable dosage forms by conventional methods known to those of skill in the art.

A lipid component of the present invention may be given alone to the treat a skin disease or skin condition. Alternatively, the lipid component may be given in combination with one or more other treatments or drugs suitable for treating the skin disease or skin condition, such as corticosteroids, calcineurin inhibitors and vitamin D. For instance, the lipid component can be administered prior to, in conjunction with (including simultaneously with), or after the other treatment or drug. In the case of another drug, the drug and the lipid component may be administered in separate pharmaceutical compositions or as part of the same pharmaceutical composition.

As used herein, the term “consisting essentially of” or “consists essentially of” excludes additional components that would affect the ability of the composition of the present invention to treat the skin disease or condition, including treating inflammation or wound healing.

The subject or animal of the present can be a mammal and preferably can be a human.

The invention also provides for a lipid fraction. The lipid fraction can be extracted from Chamaecyparis obtusa as well as from other plants and/or trees including but not limited to members of the Cupressaceae (cypress) family, Taxodiaceae family, and Sciadopityaceae family. This lipid fraction, can also be synthetically produced. The lipid fraction includes one or more of the following compounds alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, and tatarol, 2-(hexylthiol)decanal.

The invention also provides for a kit comprising a lipid component of the present invention.

As used herein, the term “consisting essentially of” excludes additional components that would affect the ability of the composition of the present invention to treat the skin disease or condition, including controlling and/or treating inflammation or wound healing.

The following experimental results are provided for purposes of illustration and are not intended to limit the scope of the invention.

### EXAMPLES

### Example 1

The following example describes the extraction of the Chamaecyparis obtusa lipid component by a supercritical fluid extraction method, starting with leaves from Chamaecyparis obtusa. In addition, as discussed in the various examples that follow, the lipid component or mixture can also be synthesized and/or combined synthetically.

The supercritical carbon dioxide extraction system and components were acquired from ILSHIN (South Korea) series supercritical fluid extractor, and included the following: 500 ml extraction vessel, temperature control unit, high-pressure pump, back pressure regulator. The independent variables were pressure (10 MPa to 45 MPa), temperature (35° C. to 70° C.), CO₂ flow rate (10 mL/min to 60 mL/min). Before the liquid CO₂ was passed into the extraction vessel, it was pressurized to the desired pressure (15 MPa) and heated to the desired temperature (40° C.). The powdered materials (200 g) were placed in the extractor vessel. The supercritical CO2 flow rate was maintained at 30 mL/min and the dynamic extraction time was fixed to 150 min During the dynamic extraction time, CO₂ carrying the crude extract flowed out of the extraction vessel unit and into a collection vessel.

The resulting lipid component/mixture as determined by gas chromatography mass spectrometry comprised the compounds listed in Table 1 along with the peak number, retention time (RT) and peak area percentage and is greater than 95% pure.

TABLE 1

| Peak | | | |
|---|---|---|---|
| Number | RT | Compound Name | Area % |
| 1 | 9.621 | α-thujene | 0.44 |
| 2 | 9.954 | α-pinene | 1.05 |
| 3 | 10.737 | camphene | 0.45 |
| 4 | 11.899 | sabinene | 9.00 |
| 5 | 12.767 | β-pinene | 2.00 |
| 6 | 14.103 | α-terpinene | 0.08 |
| 7 | 14.513 | benzene | 0.38 |
| 8 | 14.736 | limonene | 1.09 |
| 9 | 16.235 | peltay3-carene | 1.60 |
| 10 | 16.882 | trans sabinene hydrate | 0.11 |
| 11 | 17.591 | terpinolene | 0.62 |
| 12 | 22.357 | 3-cyclohexen-1-ol | 0.46 |
| 13 | 22.355 | terpinene-4-ol | 0.11 |
| 14 | 23.099 | 1,2-benzenediol | 0.17 |
| 15 | 25.670 | linalyl acetate | 0.41 |
| 16 | 27.343 | borneol | 0.57 |
| 17 | 27.343 | bornyl acetate | 0.18 |
| 18 | 27.338 | α-thujone | 0.18 |
| 19 | 30.063 | terpinyl acetate | 20.39 |
| 20 | 31.976 | isolongifolene | 0.12 |
| 21 | 33.770 | Widdrene (also referred to as thujopsene) | 3.11 |
| 22 | 34.898 | epi-bicyclosesquiphellandrene | 1.60 |
| 23 | 35.094 | α-humulene | 0.11 |
| 24 | 38.497 | guaiol | 18.44 |
| 25 | 38.497 | elemol | 18.44 |
| 26 | 40.766 | cedrol | 1.33 |
| 27 | 42.505 | β-eudesmol | 1.06 |
| 28 | 48.308 | rosifoliol | 0.95 |
| 29 | 50.975 | rimuene | 0.53 |
| 30 | 52.115 | hexadecanoic acid (also referred to as | 4.63 |
| | | palmitic acid) | |
| 31 | 52.115 | cembrene | 2.95 |
| 32 | 53.065 | hexadecanoic acid | 0.19 |
| 33 | 54.387 | verticellol | 0.85 |
| 34 | 62.537 | totarol | 0.29 |
| 35 | 63.700 | totara-7 | 0.43 |
| 36 | 63.782 | 9-octacecenamide | 2.68 |
| 37 | 63.782 | tatarol | 2.74 |
| 38 | 69.635 | 2-(hexylthio)decanal | 0.23 |

### Example 2

### Methods for Examples 2-10

### Normal Human Keratinocytes Culture:

Normal human keratinocytes were grown in serum-free cell culture medium (EpiLife cell culture medium from Cascade Biologics, Portland, Oreg., USA) containing 0.06 mmol/L calcium chloride, 1% human keratinocytes growth supplement V2 (Cascade Biologics), and 1% antibiotics (penicillin/streptomycin) under standard tissue culture conditions. To demonstrate the effects of a lipid component or mixture from Table 1 on expression of both filaggrin and HBD-3, keratinocytes were seeded at 2×10⁵ cells per well in a 24-well plate and differentiated with 0.06 mmol/L or 1.3 mmol/L CaCl₂ for 2 days, and then the cells were stimulated with various concentrations of the lipid component/mixture or a polysaccharide Chamaecyparis obtusa extract for an additional 2 days.

To further demonstrate that the lipid component/mixture of Table 1 overcomes the inhibitory effects of IL-4 and IL-13 on both filaggrin and HBD-3, keratinocytes were incubated with 50 ng/mL of IL-4 and 50 ng/mL of IL-13 for a day, and then the cells were continuously stimulated with IL-4 and IL-13 in the presence or absence of 0.001% of the lipid component/mixture of Table 1 for an additional 2 days.

### RNA Preparation and Real Time RT-PCR

Total RNA was isolated from keratinocytes by using RNeasy kits according to the manufacturer's guidelines (Qiagen, Valencia, Calif., USA) for real-time RT-PCR. One microgram RNA was reverse-transcribed in a 20-4, reaction containing Random Primers (Invitrogen, Carlsbad, Calif., USA), RNase Inhibitor (Invitrogen), and Superscript III enzyme (Invitrogen) for 60 minutes at 42° C. and then 70° C. for 15 minutes. Real-time RT-PCR was performed and analyzed by a dual-labeled fluorigenic probe method using an ABI Prism 7700 sequence detector (Applied Biosystems, Foster City, Calif., USA). Relative expression levels were calculated by the relative standard curve method as outlined in the manufacturer's technical bulletin. A standard curve was generated using the fluorescent data from the 10-fold serial dilutions of total RNA of the highest expression sample. This was then used to calculate the relative amounts of target mRNA in test samples. Quantities of all targets in test samples were normalized to the corresponding 18s RNA transcript in cultured keratinocytes. Primers and probes for human 18S, filaggrin and HBD-3 were purchased from Applied Biosystems.

### Statistical Analyses

All statistical analysis was conducted with GraphPad Prism software (GraphPad Software, La Jolla, Calif., USA). Comparison of expression levels were performed by using one-way analysis of variance (ANOVA) and significant differences were determined by a Tukey-Kramer test. In case where two groups were compared, data were analyzed using an unpaired T test.

### Example 3

The following example demonstrates the induction of filaggrin gene expression in undifferentiated human keratinocytes treated with a lipid component/mixture of Table 1.

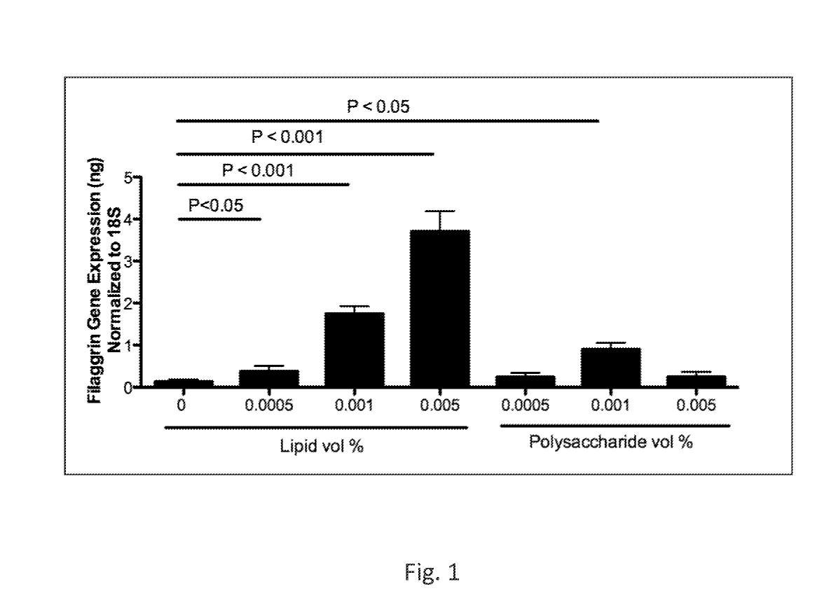

Undifferentiated human keratinocytes were cultured as described in Example 2. The cells were stimulated with various concentrations of the lipid component or a polysaccharide Chamaecyparis obtusa extract as described in Example 2 and FIG. 1. Total RNA was isolated from keratinocytes as described in Example 2. Expression levels of filaggrin gene expression were determined by Real-time RT-PCR as described in Example 2 and normalized to the corresponding 18s RNA transcript in the cultured keratinocytes. The results are presented in FIG. 1.

### Example 4

The following example demonstrates the induction of filaggrin gene expression in differentiated human keratinocytes treated with the lipid component/mixture of Table 1.

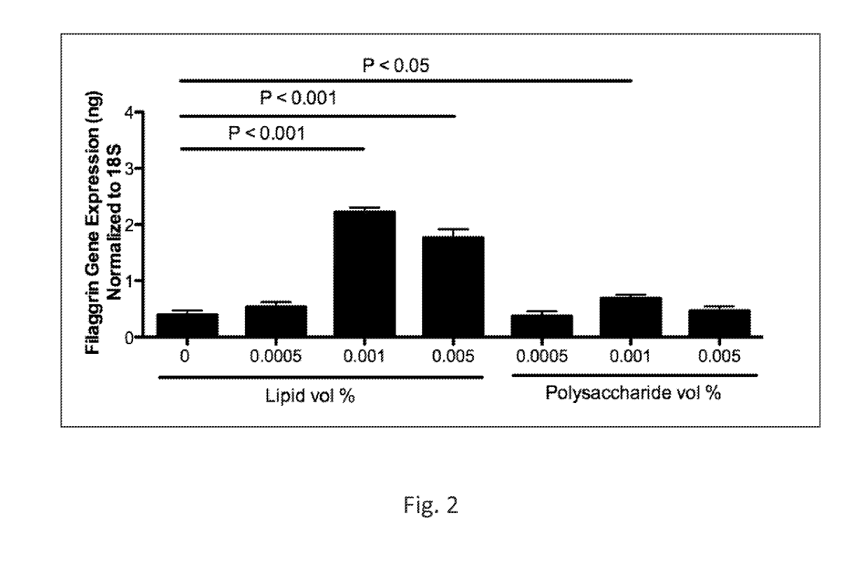

Differentiated human keratinocytes were cultured as described in Example 2. The cells were stimulated with various concentrations of the lipid component/mixture or a polysaccharide Chamaecyparis obtusa extract as described in Example 2 and FIG. 2. Total RNA was isolated from keratinocytes as described in Example 2. Expression levels of filaggrin gene expression were determined by Real-time RT-PCR as described in Example 2 and normalized to the corresponding 18s RNA transcript in the cultured keratinocytes. The results are presented in FIG. 2.

### Example 5

The following example demonstrates the induction of human beta-defensin-3 gene expression in undifferentiated human keratinocytes treated with the lipid component/mixture of Table 1.

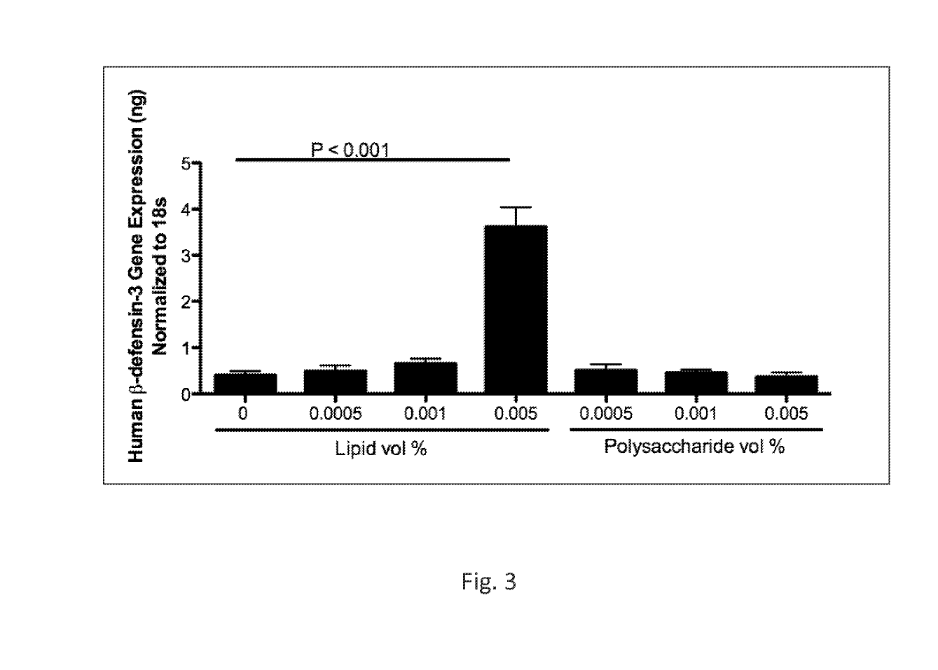

Undifferentiated human keratinocytes were cultured as described in Example 2. The cells were stimulated with various concentrations of the lipid component/mixture or a polysaccharide Chamaecyparis obtusa extract as described in Example 2 and FIG. 3. Total RNA was isolated from keratinocytes as described in Example 2. Expression levels of human beta-defensin-3 gene expression were determined by Real-time RT-PCR as described in Example 2 and normalized to the corresponding 18s RNA transcript in the cultured keratinocytes. The results are presented in FIG. 3.

### Example 6

The following example demonstrates the induction of human beta-defensin-3 gene expression in differentiated human keratinocytes treated with the lipid component/mixture of Table 1.

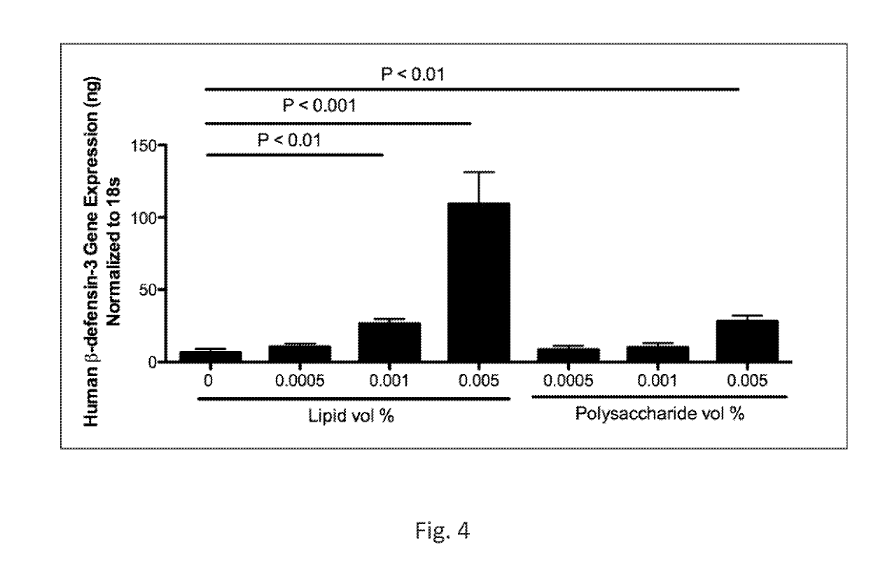

Differentiated human keratinocytes were cultured as described in Example 2. The cells were stimulated with various concentrations of the lipid component/mixture of Table 1 or a polysaccharide Chamaecyparis obtusa extract as described in Example 2 and FIG. 4. Total RNA was isolated from keratinocytes as described in Example 2. Expression levels of human beta-defensin-3 gene expression were determined by Real-time RT-PCR as described in Example 2 and normalized to the corresponding 18s RNA transcript in the cultured keratinocytes. The results are presented in FIG. 4.

### Example 7

The following example demonstrates that 0.001% of the lipid component/mixture of Table 1 overcomes the inhibitory effects of Th2 cytokines on filaggrin expression in undifferentiated keratinocytes.

Undifferentiated human keratinocytes were cultured as described in Example 2 and further were incubated with 50 ng/mL of IL-4 and 50 ng/mL of IL-13 for a day, and then the cells were continuously stimulated with IL-4 and IL-13 in the presence or absence of 0.001% of the lipid component/mixture for an additional 2 days.

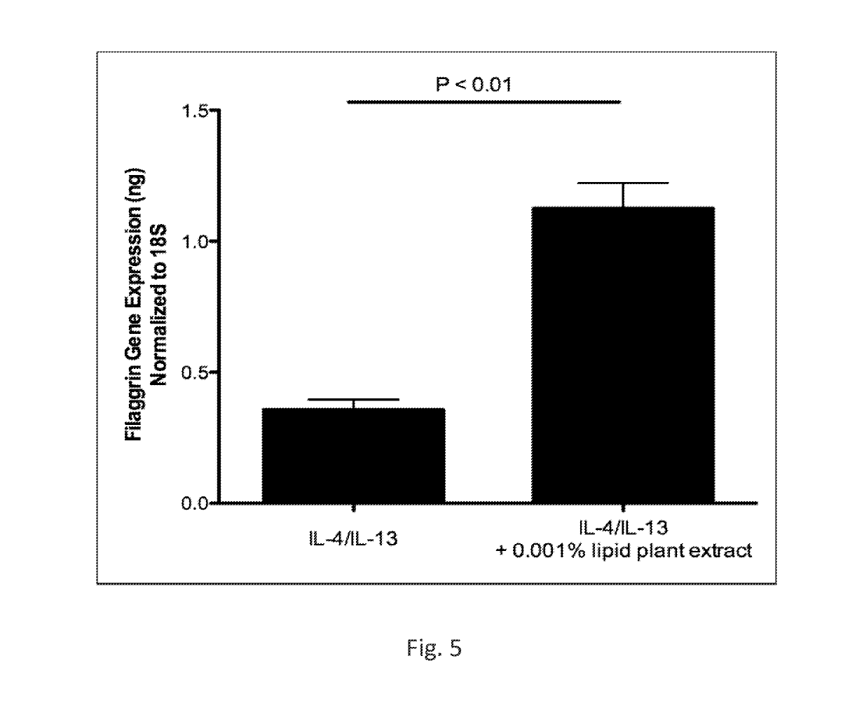

Total RNA was isolated from the keratinocytes as described in Example 2. Expression levels of filaggrin gene expression were determined by Real-time RT-PCR as described in Example 2 and normalized to the corresponding 18s RNA transcript in the cultured keratinocytes. The results are presented in FIG. 5.

### Example 8

The following example demonstrates that 0.001% of the lipid component/mixture of Table 1 overcomes the inhibitory effects of Th2 cytokines on filaggrin expression in differentiated keratinocytes.

Differentiated human keratinocytes were cultured as described in Example 2 and further were incubated with 50 ng/mL of IL-4 and 50 ng/mL of IL-13 for a day, and then the cells were continuously stimulated with IL-4 and IL-13 in the presence or absence of 0.001% of the lipid component/mixture of Table 1 for an additional 2 days.

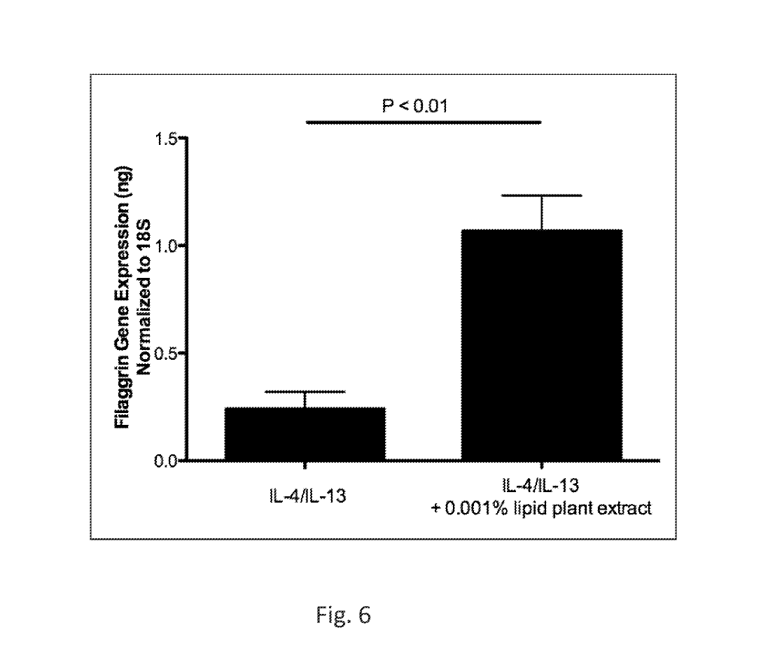

Total RNA was isolated from the keratinocytes as described in Example 2. Expression levels of filaggrin gene expression were determined by Real-time RT-PCR as described in Example 2 and normalized to the corresponding 18s RNA transcript in the cultured keratinocytes. The results are presented in FIG. 6.

### Example 9

The following example demonstrates that 0.001% of the lipid component/mixture of Table 1 overcomes the inhibitory effects of Th2 cytokines on human beta-defensin expression in differentiated keratinocytes.

Differentiated human keratinocytes were cultured as described in Example 2 and further were incubated with 50 ng/mL of IL-4 and 50 ng/mL of IL-13 for a day, and then the cells were continuously stimulated with IL-4 and IL-13 in the presence or absence of 0.001% of the lipid component/mixture for an additional 2 days.

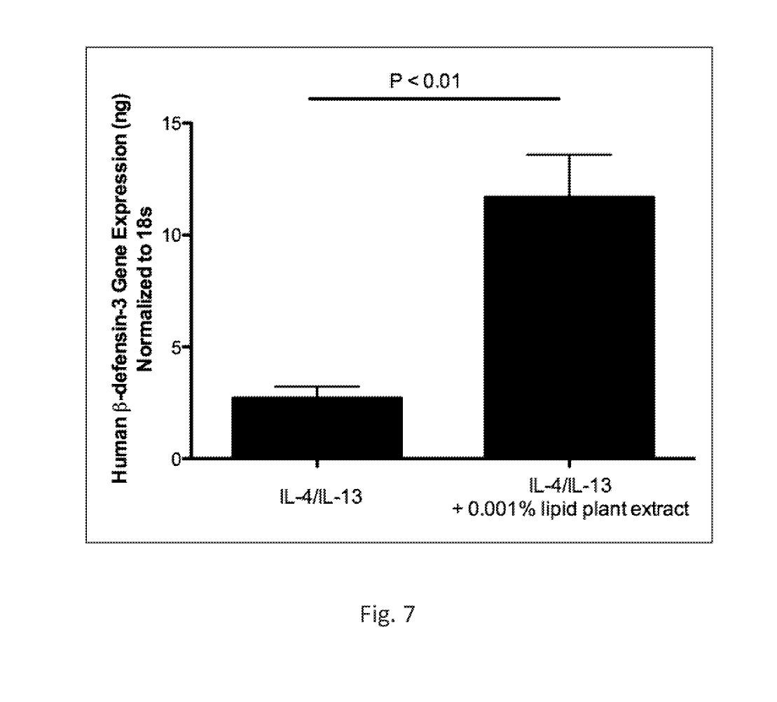

Total RNA was isolated from the keratinocytes as described in Example 2. Expression levels of human β-defensin-3 gene expression were determined by Real-time RT-PCR as described in Example 2 and normalized to the corresponding 18s RNA transcript in the cultured keratinocytes. The results are presented in FIG. 7.

### Example 10

The following example shows that a Chamaecyparis obtusa lipid component does not have keratinocyte toxicity.

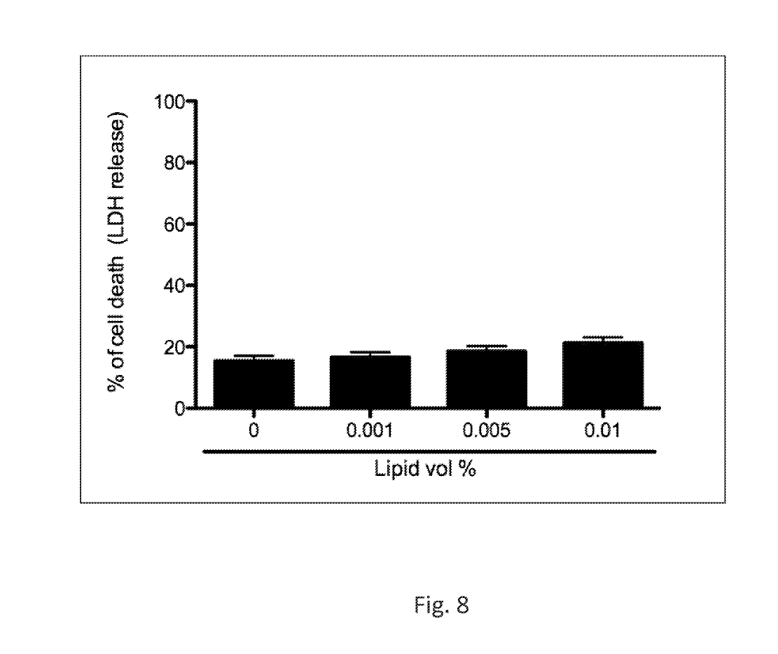

A lactate dehydrogenase assay was performed wherein keratinocytes were plated in quadruplicate at 20,000 per well in a 96-well plate and allowed to adhere overnight. Cells were incubated in the 0.06 mmol/L of CaCl₂ with various concentrations of a Chamaecyparis obtusa lipid component for 2 days. Lactate dehydrogenase (LDH) release was quantitated by using the Cyto-Tox One Kit from Promega (Madison, Wis., USA) according to the manufacturer's instructions. The results are presented in FIG. 8.

### Example 11

### Methods for Examples 12-15

### Normal Human Keratinocyte Culture

Normal human keratinocytes were grown as described in Example 2. For demonstrating the effects of a purified lipid mixture comprising terpinyl acetate, guaiol, elemol, sabinene, palmitic acid (also referred to as hexadecanoic acid), thujopsene (also referred to as widdrene), totarol, 9-octadecenamide, β-pinene and cembrene, on expression of both filaggrin and HBD-3, keratinocytes were seeded at 2×10⁵ cells per well in a 24-well plate and differentiated with 1.3 mmol/L CaCl₂ for 2 days, and then the cells were stimulated with various concentrations of the purified lipid mixture, for an additional 2 days. To further demonstrate that the same purified lipid mixture overcomes the inhibitory effects of IL-4 and IL-13 on both filaggrin and HBD-3, keratinocytes were incubated with 50 ng/mL of IL-4 and 50 ng/mL of IL-13 for a day, and then the cells were continuously stimulated with IL-4 and IL-13 in the presence or absence of 0.02% of the purified lipid mixture for an additional 2 days.

As discussed in the various examples that follow, the lipid component or mixture can be synthesized and/or combined synthetically or extracted as discussed above.

### RNA Preparation and Real Time RT-PCR

Total RNA was isolated from keratinocytes as described in Example 2. Real-time RT-PCR was performed and analyzed as described in Example 2.

### Bactericidal Assay:

Experiments were conducted using methicillin sensitive Staphylococcus aureus (MSSA, ATCC 29213) and methicillin resistant Staphylococcus aureus (MRSA, ATCC BAA-1556). Staphylococci were grown overnight in Tryptic Soy Broth (Becton Dickinson, Franklin Lakes, N.J.) at 37° C. and reseeded in fresh media three hours prior to experiment start. The bacterial concentration was determined by optical density. Twenty-four hours before the experiment, the cell culture media was removed and the cultures were rinsed 3 times with 37° C. KGM. The media was replaced with antibiotic-free KGM containing all supplements and 1.3 mmol/L CaCl₂. At experiment start, 1×10⁶ bacteria/mL were inoculated into each well in fresh antibiotic-free medium. Controls received media alone. Cells were co-incubated with bacteria at 37° C., 5% CO₂ for 3 hours. Lysed, viable, and total bacteria were counted.

### Lactate Dehydrogenase Assay

For the lactate dehydrogenase (LDH) assay, keratinocytes were plated in quadruplicate at 20,000 per well in a 96-well plate and allowed to adhere overnight. Cells were incubated in the 0.06 mmol/L of CaCl₂ with various concentrations of the purified lipid mixture noted above for 2 days. LDH release was quantitated (Cyto-Tox One Kit from Promega from Madison, Wis., USA) according to the manufacturer's instructions.

### Statistical Analyses

All statistical analysis was conducted as described in Example 2.

### Example 12

This example demonstrates the induction of filaggrin gene expression and HBD-3 gene expression in undifferentiated human keratinocytes treated with a lipid mixture comprising terpinyl acetate, guaiol, elemol, sabinene, palmitic acid (also referred to as hexadecanoic acid), thujopsene (also referred to as widdrene), totarol, 9-octadecenamide, β-pinene and cembrene.

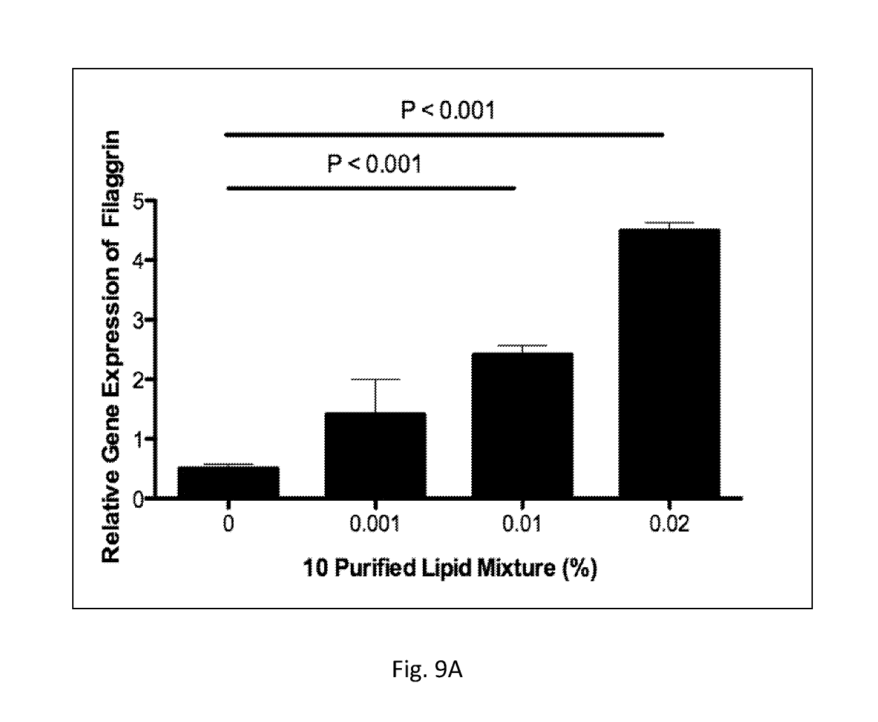

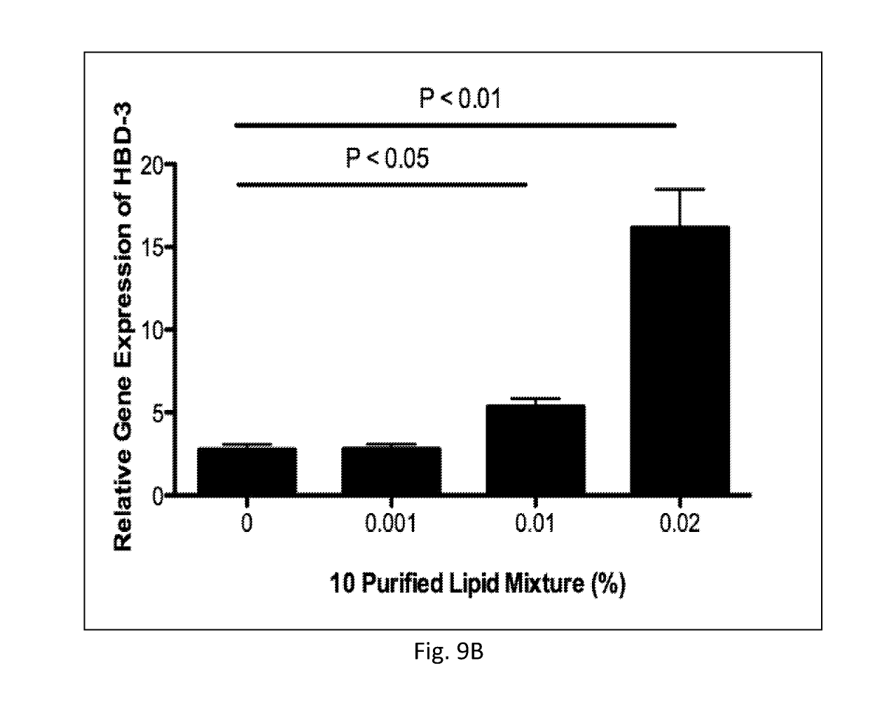

Human primary keratinocytes were stimulated with various concentrations of the purified lipid mixture for 2 days. The gene expression of filaggrin and HBD-3 was examined using real-time RT-PCR. Filaggrin gene expression was significantly (P<0.001) induced by the purified lipid mixture with concentration as low as 0.01% compared with media alone (FIG. 9A). HBD-3 gene expression was significantly (P<0.05) induced by the purified lipid mixture with concentration as low as 0.01% compared with media alone (FIG. 9B).

### Example 13

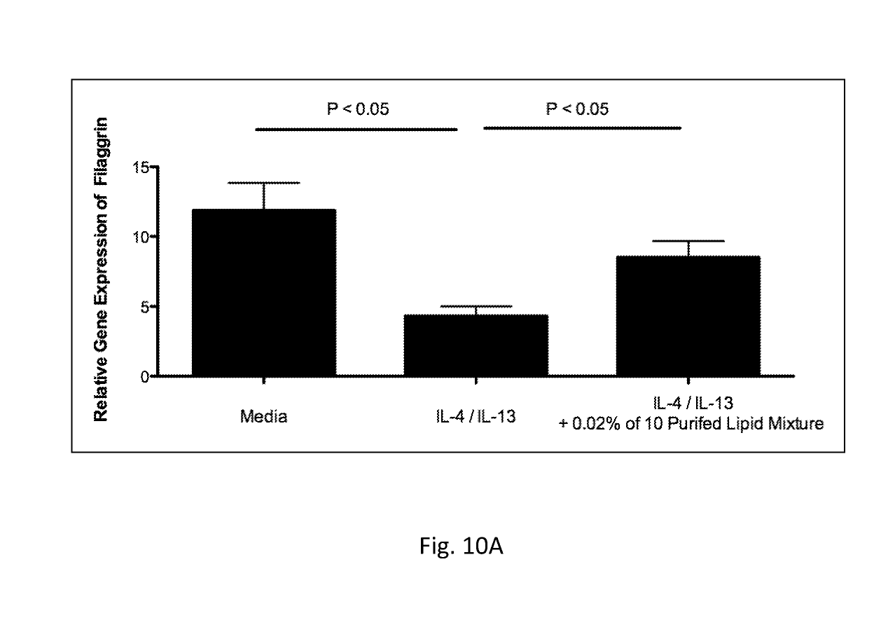

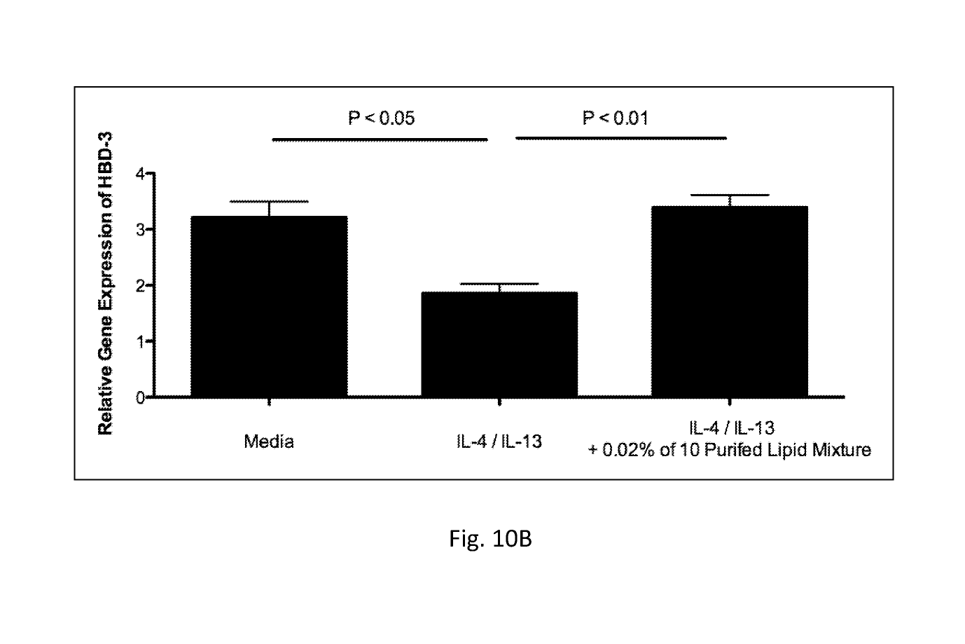

This example demonstrates that the lipid mixture described in Example 12 overcomes inhibitory effects of Th2 cytokines on filaggrin and HBD-3. Human primary keratinocytes were incubated with 50 ng/mL of IL-4 and 50 ng/mL of IL-13 for a day, and then the cells were continuously stimulated with IL-4 and IL-13 in the presence or absence of 0.02% of the purified lipid mixture for an additional 2 days. The gene expression of filaggrin and HBD-3 was examined using real-time RT-PCR. Filaggrin gene expression was significantly (P<0.05) increased in keratinocytes treated with a combination of Th2 cytokines and 0.02% of 10 purified lipid mixture compared with keratinocytes treated Th2 cytokines alone (FIG. 10A). HBD-3 gene expression was significantly (P<0.01) increased in keratinocytes treated with a combination of Th2 cytokines and 0.02% of the purified lipid mixture compared with keratinocytes treated Th2 cytokines alone (FIG. 10B).

### Example 14

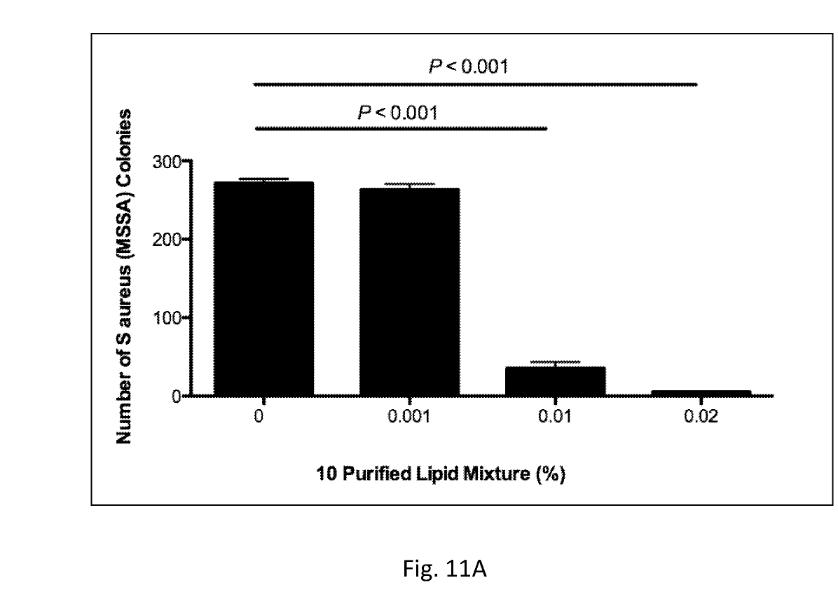

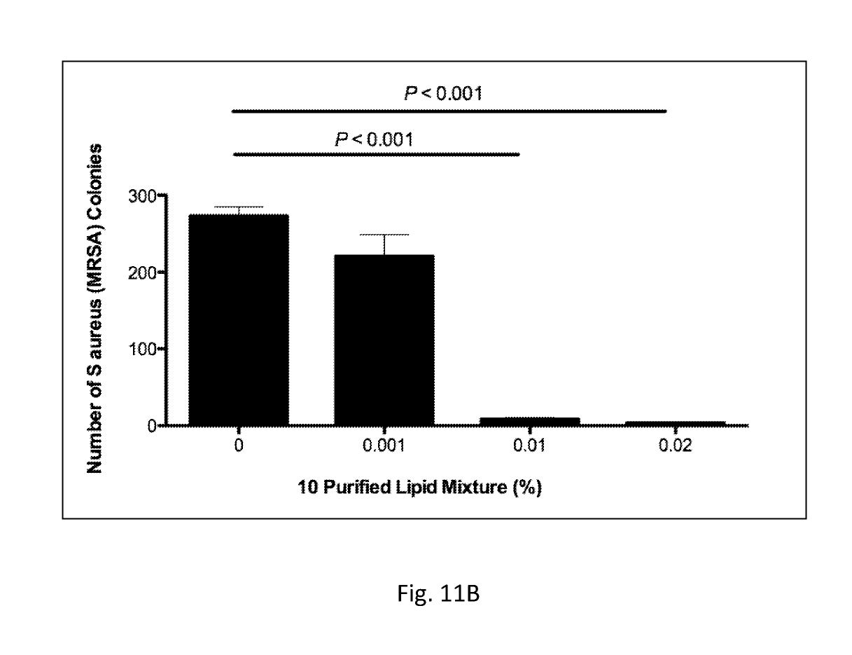

This example demonstrates that the purified lipid mixture described in Example 12 inhibits Staphylococcus aureus. Inhibitory effects of the lipid mixture on bacteria were analyzed using the bactericidal assay described in Example 11. Methicillin sensitive S aureus (MSSA) was significantly (P<0.001) inhibited by the purified lipid mixture with concentration as low as 0.01% (FIG. 11A). Methicillin resistant S aureus (MRSA) was significantly (P<0.001) inhibited by the purified lipid mixture with concentration as low as 0.01% (FIG. 11B).

### Example 15

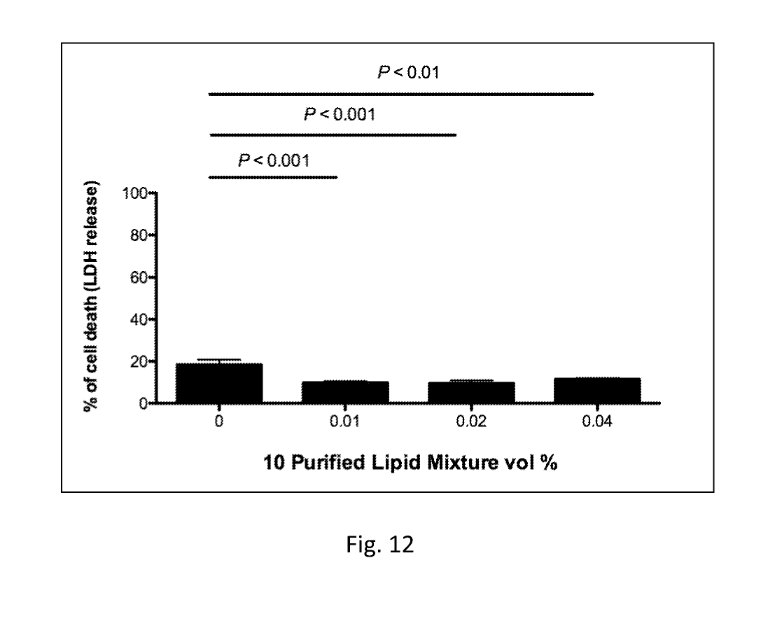

This example demonstrates that the purified lipid mixture described in Example 12 is not toxic to human primary keratinocytes, and has a protective effect for keratinocyte. The toxicity of the lipid mixture on keratinocytes was evaluated with the lactate dehydrogenase (LDH) assay described in Example 11. LDH release was not increased by the purified lipid mixture as high as 0.04%, but reduced by various concentrations of the purified lipid mixture (FIG. 12).

### Example 16

This example describes treating an inflammatory skin disease or condition, such as atopic dermatitis, with a composition comprising a lipid component as described herein.

A lipid component comprising one or more of the following lipids alpha-thujene, alpha-pinene, camphene, sabinene, beta-pinene, alpha-terpinene, benzene, limonene, peltay3-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal and combinations thereof is administered by topical administration to a subject having an inflammatory skin disease or condition, such as atopic dermatitis. The gene expression of filaggrin and HBD-3 is determined and is significantly increased in the skin of the subject having the inflammatory skin disease or condition as compared to the skin of a subject having the same inflammatory skin disease or condition that is treated with a vehicle lacking the lipid component. Skin inflammation is reduced in the skin of the subject having been administered the lipid component or mixture. In addition reduction of Staphylococcus aureus infection is expected to be reduced.

Various combinations of the lipids can be combined and administered to the subject such as a lipid component comprising terpinyl acetate, guaiol, elemol, sabinene, palmitic acid (also referred to as hexadecanoic acid), thujopsene (also referred to as widdrene), totarol, 9-octadecenamide, β-pinene and cembrene. The lipid component can be purified.

### Example 17

This example describes accelerating wound healing in a subject having diabetes with a composition comprising a lipid component as described herein.

A lipid component comprising one or more of the following lipids alpha-thujene, alpha-pinene, camphene, sabinene, beta-pinene, alpha-terpinene, benzene, limonene, peltay3-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal and combinations thereof is administered to a wound in a subject in need of wound healing. Upon administration of the lipid component to the subject would healing is significantly accelerated in a wound treated with the lipid component as compared to a wound of a similar subject that is treated with a vehicle lacking the lipid component. Accelerated wound healing is characterized by a shortened time to wound closure. In one aspect, the wound may be super-infected with Staphylococcus aureus or other bacteria and thus administration of the lipid component to this wound will kill the bacterial pathogens in the wounds.

All references cited herein are incorporated by reference in their entireties.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following exemplary claims.

What is claimed is:

1. A method of treating a skin disease or condition in an animal, the method comprising administering to the animal a composition comprising a lipid component selected from the group consisting of alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal and combinations thereof, as an active ingredient.

2. The method of claim 1 wherein the skin disease or condition is an inflammatory skin disease or condition.

3. The method of claim 2 wherein the inflammatory skin disease or condition is selected from the group consisting of atopic dermatitis, contact dermatitis, seborrhoic dermatitis, and acne.

4. The method of claim 3, wherein the inflammatory skin disease or condition is atopic dermatitis.

5. The method of claim 1 wherein the skin disease or condition is selected from the group consisting of xeroderma, psoriasis, and ichthyosis.

6. The method of claim 1, wherein the skin disease or condition is a skin wound.

7. The method of claim 1, wherein the lipid component comprises terpinyl acetate, guaiol, elemol, sabinene, palmitic acid, thujopsene, totarol, 9-octadecenamide, β-pinene and cembrene.

8. The method of claim 1, wherein the lipid component consists essentially of terpinyl acetate, guaiol, elemol, sabinene, palmitic acid, thujopsene, totarol, 9-octadecenamide, β-pinene and cembrene.

9. The method of claim 1, wherein the lipid component consists of terpinyl acetate, guaiol, elemol, sabinene, palmitic acid, thujopsene, totarol, 9-octadecenamide, 3-pinene and cembrene.

10. The method of claim 1, wherein the lipid component induces expression of filaggrin.

11. The method of claim 1, wherein the lipid component induces expression of beta-degensin-3 (HBD-3).

12. The method of claim 1, wherein the lipid component attenuates the inhibitory effect of proinflammatory cytokines on filaggrin expression, wherein the proinflammatory cytokine is a Th2 cytokine selected from the group consisting of IL-4, IL-13, IL-25, IL-31, and IL-33.

13. The method of claim 1, wherein the lipid component attenuates the inhibitory effect of proinflammatory cytokines on beta-defensin-3 expression, wherein the proinflammatory cytokine is a Th2 cytokine selected from the group consisting of IL-4, IL-13, IL-25, IL-31, and IL-33.

14. The method of claim 1, wherein the lipid component is non-toxic and protects keratinocytes.

15. The method of claim 1 wherein the composition is administered topically or transdermally.

16. The method of claim 1, wherein the composition is in a form selected from the group consisting of a solution, a gel, a solid, an emulsion, a suspension, a microemulsion, microcapsules, microgranules, ionic (liposome) and non-ionic vesicles, cream, skin lotion, an ointment, powder, a spray, a conceal stick, foam and aerosol.

17. A method of inducing expression of filaggrin in keratinocytes of an animal in need thereof comprising administering to the animal a composition comprising a lipid component selected from the group consisting of alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal and combinations thereof as an active ingredient, wherein the lipid component induces expression of filaggrin.

18. A method of inducing expression of beta-defensin-3 in keratinocytes of an animal in need thereof comprising administering to the animal a composition comprising a lipid component elected from the group consisting of alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal and combinations thereof as an active ingredient, wherein the lipid component induces expression of beta-defensin.

19. A method for preventing a skin disease or condition in an animal, the method comprising administering to the animal a composition comprising a lipid component selected from the group consisting of alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal and combinations thereof as an active ingredient and wherein the skin disease or condition is skin cancer or skin burn from UV exposure.

20. A pharmaceutical composition comprising a pharmaceutically-acceptable carrier, and a lipid component selected from the group consisting of alpha-thujene, alpha-pinene, camphene, sabinene, beta-pinene, alpha-terpinene, benzene, limonene, peltay2-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal and combinations thereof.

21. A lipid fraction wherein the fraction comprises one or more of the following lipids selected from the groups consisting of alpha-thujene, alpha-pinene, camphene, sabinene, beta-pinene, alpha-terpinene, benzene, limonene, peltay3-carene, trans sabinene hydrate, terpinolene, 3-cyclohexen-1-ol, terpinene-4-ol, 1,2-benzenediol, linalyl acetae, borneol, bornyl acetate, alph-thujone, terpinyl acetate, isolongifolene, epit-bicyclosesquiphellandrene, alpha-humulene, guaiol, elemol, cedrol, beta-eudesmol, rosifoliol, rimuene, hexadecanoic acid, cembrene, verticellol, totarol, totara-1,9-octadecenamide, tatarol, 2-(hexylthiol)decanal and combinations thereof.

22. The lipid fraction of claim 21 wherein the lipid fraction comprises one or more of the following lipids selected from the group consisting of terpinyl acetate, guaiol, elemol, sabinene, palmitic acid, thujopsene, totarol, 9-octadecenamide, β-pinene and cembrene.

23. The lipid fraction of claim 21, wherein one or more of the lipids are synthetically produced.

24. The lipid fraction of claim 21 wherein one or more of the lipids are extracted from the leaves of Chamaecyparis obtusa.
